(19) United States
(12) Patent Application Publication
(10) Pub. No.: US 2009/0317339 A1
(43) Pub. Date: Dec. 24, 2009

(54) Teeth Bleaching Compositions and Devices

(76) Inventors: Deepak Sharma, Flemington, NJ (US); Robert J. Gambogi, Hillsborough, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/144,211

(22) Filed: Jun. 23, 2008

(51) Int. Cl. A61K 8/18; A61K 8/22; A61Q 11/00

(52) U.S. Cl. 424/49; 424/53; 424/401

(57) ABSTRACT

The present invention is directed to an oral care device configured to contact teeth, where the device includes disposed therein a bioactive glass in an amount effective to mineralize teeth when contacted with the teeth under conditions effective to mineralize the teeth; and a tooth-bleaching agent in an amount effective to whiten teeth when contacted with the teeth under conditions effective to whiten the teeth, and to methods for simultaneously mineralizing and whitening teeth utilizing the devices of the present invention.

### FIELD OF THE INVENTION

This invention relates to oral care compositions and devices that can simultaneously mineralize and whiten teeth, reduce sensitivity and/or restore or build mineral deposits.

### BACKGROUND OF THE INVENTION

Patient applied tooth whitening was popularized following publication of the clinical observed efficacy of 10 percent carbamide peroxide in custom trays. Dental dispensed systems predominated for the next decade until the launch of OTC products in the early 2000s. A major side effect of teeth whitening products is whitening induced tooth sensitivity. Although transient in nature it has been frequently reported for products dispensed at the dentist office as well as for products available through mass retail. The incidences of whitening induced tooth sensitivity have been reported to increase with increasing concentration of hydrogen peroxide and/or increased frequency of use. Formulation type, product form and delivery device can also be contributing factors in these side effects.

Moreover, dental caries is initiated by the demineralization of hard tissue on teeth by organic acids. A substantial number of mineral ions can be removed from the hydroxyapatite (HAP) lattice network of tooth enamel without destroying its structural integrity; however, such demineralized enamel leads to the formation of tiny lesions that transmit hot, cold, pressure and pain more readily than normal enamel, and such lesions are also thought to be precursors to dental caries formation. The incidences of peroxide induced teeth sensitivity may be much higher in such individuals.

Currently, there are products on the market that claim to mitigate whitening induced sensitivity. However, the data to support these products are limited. The two common approaches that are currently used to mitigate classical teeth sensitivity and are also used for whitening induced sensitivity are: 1) desensitization of nerves using a KNO₃ and 2) occlusion of exposed dentinal tubules by compounds that can deliver calcium, phosphate, or fluoride ions and precipitate on the surface.

As whitening induced sensitivity is a transient side effect and has not been completely understood, the approaches and treatment protocols used to mitigate classic dentinal sensitivity may not be completely effective. Moreover, KNO₃ requires at least 2 weeks of continuous application to be effective in reducing teeth sensitivity issues. For the occluding technology, the need to deliver effective concentration and chemistry has always been a challenge.

Recently, amorphous calcium phosphate (ACP) and its fluoride (ACPF) and carbonate-containing (ACCP) derivatives have been used to mineralize and to desensitize teeth. ACP, when exposed and/or dissolved in water, rapidly reverts to a more thermodynamically stable form, e.g., hydroxyapatite (HAP), which is substantially insoluble and, thus, calcium and phosphate ions are not biologically available. Some have solved this reversion problem by providing either two part or dual delivery systems.

Bioactive glasses have also been used in anhydrous dentifrices formulations. The aqueous instability of bioactive glasses limits their use and effectiveness in oral care compositions.

The desire to reduce transient teeth sensitivity during the teeth whitening process is known, and there is a need for products that simultaneously mineralize and whiten the teeth, reduce sensitivity and/or harden teeth.

### SUMMARY OF THE INVENTION

The present invention is directed to an oral care device configured to contact teeth, where the device includes disposed therein a bioactive glass in an amount effective to mineralize teeth when contacted with the teeth under conditions effective to mineralize the teeth; and a tooth-bleaching agent in an amount effective to whiten teeth when contacted with the teeth under conditions effective to whiten the teeth, and to methods for simultaneously mineralizing and whitening teeth utilizing the devices of the present invention.

### DESCRIPTION OF DRAWINGS

FIG. 1 is a perspective, exploded view of a three-layer dental strip;

FIG. 1A is a cross-sectional view of the dental strip of FIG. 1, taken along 1A-1A;

FIG. 2 is a cross-sectional view of dental strip having two layers;

FIG. 3 is a cross-sectional view of a dental strip having a single layer;

FIG. 4 is a perspective view of one exemplary disintegrable film product of the current invention; and

FIG. 5 is a perspective view of another exemplary disintegrable film product of the current invention, where the disintegrable film of the present invention forms the first or backing layer of a two-layer film.

### DETAILED DESCRIPTION OF THE INVENTION

Generally, the invention relates to oral care devices and compositions that can simultaneously mineralize and whiten the teeth, reduce the incidence of tooth sensitivity and/or harden teeth. Such devices and compositions comprise a bioactive glass and a tooth-bleaching agent. In some embodiments, the devices and compositions are maintained under anhydrous conditions until used in the oral cavity to maintain the bioavailability of minerals and/or the potency of the tooth-bleaching agent. In some embodiments the bioactive glass and the tooth-bleaching agent are maintained apart from one another in the oral care device such that an admixture of the components is avoided prior to application of the device to the teeth. In other embodiments the bioactive glass is admixed with the tooth-bleaching agent and disposed within the device as an admixture prior to application of the device to the teeth.

As used herein, bioactive glass means an inorganic silica-based glass material comprising, in weight percentage, between 40 and 60 percent SiO₂, between 0 and 30 percent Na₂O, between 0 and 30 percent K₂O, between 15 and 30 percent CaO, between 0 and 15 percent CaF₂, between 0 and 15 percent NaF, between 0 and 10 percent Li₂O, between 0 and 10 percent SnO, between 0 and 10 percent SrO, between 0 and 8 percent P₂O₅, between 0 and 6 percent Fe₂O₃, between 0 and 3 percent MgO, between 0 and 3 percent B₂O₃, between 0 and 3 percent Al₂O₃, and between 0 and 3 percent ZnO.

In some embodiments, the bioactive glass is Bioactive glass 45S5, containing 45 percent SiO₂, 24.5 percent CaO, 24.5 percent Na₂O, and 6 percent P₂O₅. This Bioactive glass is capable of releasing biologically relevant ions when hydrolyzed with physiological fluids. This material can be prepared in house by using melt-derived protocol or can be commercially purchased from MO-SCI Healthcare L.L.C, Missouri.

In certain embodiments the bioactive glass may be modified to enhance its properties or to otherwise provide additional advantageous properties apart from mineralization. For example, the bioactive glass may be treated to include functional species or materials. A particularly relevant functional material would be biologically-active molecules. The biologically active molecules are defined as those organic molecules having an effect in a biological system, whether such system is in vitro, in vivo, or in situ. Biologically-active molecules include, without limitation, antibacterial and anti-inflammatory agents, biofilm modulators, and cell attachment molecules. It is contemplated that many serum-protein like organic molecules will also function as biologically-active molecules. Such modifications may be accomplished by treatment of the bioactive glass with a solution to coat the bioactive glass, or any process whereby the functional material is bound to the bioactive glass.

The tooth-bleaching agent can be, for example, a peroxide, a metal chlorite, a perborate, a percarbonate, a peroxyacid, a persulfate, a carbamide, a hypochlorite, chlorine dioxide, or mixtures of these. In specific embodiments, the tooth-bleaching agent includes hydrogen peroxide, a complex thereof, or a source thereof.

In one aspect, the invention features an oral care device, such as a strip or a mouthpiece, configured to contact one or more teeth. The oral care device includes a bioactive glass and a tooth-bleaching agent. Forms of strips include films, patches, trays and/or wraps that are suitable to place on a tooth. These strips can be both dissolvable and non-dissolvable.

In one embodiment, the oral care device is in the form of a strip. The strip can include a polymeric material. The polymeric material can be selected from, but is not limited to, polyvinylpyrrolidone, a cellulosic polymer, polyvinyl alcohol, hydroxyethyl cellulose, hydroxypropyl cellulose, hydroxypropylmethyl cellulose, sodium carboxymethyl cellulose, polyethylene oxide, polyethylene glycol, polyacrylic acid, silicone, hydroxypropylmethylcellulose phthalate (HPMCP), polyvinyl acetate, cellulose acetate phthalate, gelatin sodium alginate, carbomer, polyvinyl pyrrolidone-vinyl acetate copolymer, polyalkylvinylether-maleic acid copolymer, polypropylene glycol, polyethylene glycol-polypropylene glycol copolymers, quaternized copolymers, quaternized copolymers of vinylpyrrolidone and dimethylaminoethyl methacrylate, or mixtures thereof. In some embodiments, the polymeric material is soluble or swollen by water. The polymeric can have a flexural modulus of less than 100,000 psi, or less than 25,000 psi.

In some embodiments, the strip has a plurality of layers. In a specific embodiment, the strip includes a first layer, configured to contact one or more teeth, that includes a bioactive glass and a tooth-bleaching agent, and a second barrier layer that includes a material suitable for forming a barrier. The first layer can include polyvinylpyrrolidone, while the second barrier layer can include hydroxypropylmethylcellulose phthalate (HPMCP), polyethylene, or polyvinyl acetate.

In another specific embodiment, the strip includes a first layer, configured to contact one or more teeth, that includes a tooth-bleaching agent, a second layer that includes a bioactive glass, and a third barrier layer that includes a material suitable for forming the barrier. The first and second layers can include polyvinylpyrrolidone, while the third barrier layer can include hydroxypropylmethylcellulose phthalate (HPMCP), polyethylene, or polyvinyl acetate.

In addition, any of the flavoring agents, fragrances, sweeteners, coloring agents, peroxide activators, enzymes, malodor controlling agents, cleaning agents, stain prevention agents, antibacterial agents, antigingivitis agents, anti-caries agents, anti-periodontitis agents and tooth sensitivity agents can be used in any of the compositions.

In another aspect, the invention features a method of treating teeth. The method includes contacting teeth to be whitened with an oral care device, e.g. a strip, that includes a bioactive glass and a tooth-bleaching agent. The bioactive glass and tooth-bleaching agent are maintained in contact with the teeth under conditions effective to whiten and mineralize the teeth, which as used herein means that the tooth whitening composition is in contact with the teeth for a sufficient period of time, whether upon a single application or multiple applications over a specified time interval or frequency, such that whitening and mineralization of the teeth is achieved. For example, compositions may remain in contact with the teeth for greater than about 30 seconds, in another embodiment from about 2 minutes to about 12 hours (e.g. overnight treatment), in another embodiment from about 3 minutes to about 120 minutes, in yet another embodiment from at least about 5 minutes to about 40 minutes, per application. In certain embodiments, application of additional strips to the teeth may be repeated in one day, for example from 2 to about 7 times per day. Additionally, the length of treatment to achieve the desired benefit, for example, tooth whitening, may last from about one day to about six months. For example, in one embodiment the method may be repeated over a period of time from one day to about 28 days, and in another embodiment from about 7 to about 28 days. The optimal duration and frequency of application will depend on the desired effect, the severity of any condition being treated, the health and age of the user and like considerations. In some embodiments, sufficient time is less than 180 minutes, less than 120 minutes, less than 60 minutes, less than 30 minutes, less than 10 minutes or even less than 5 minutes. As with whitening efficacy, the greater the contact time, the greater the potential to mineralize teeth, which should subsequently mitigate whitening-induced sensitivity. The method can be used to simultaneously mineralize and whiten teeth, treat tooth sensitivity, harden teeth and/or restore or build minerals.

In another aspect, the invention features a method of simultaneously mineralizing and whitening teeth with reduced incidence of whitening-induced tooth sensitivity. The method includes contacting one or more teeth with a device that includes a bioactive glass, and a tooth-bleaching agent. Contact is maintained with the one or more teeth such that the bioactive glass and the tooth-bleaching agent are in contact with the tooth surface to be treated for a time to at least whiten and partially mineralize the one or more teeth. This composition will reduce the incidence of transient teeth sensitivity that is often associated with teeth bleaching.

To further enhance the activity of this composition, other calcium, potassium, oxalate and phosphate salts, as well as hydroxyapatite (HAP) material, can also be included in the composition used in devices of the present invention. Examples include potassium oxalate, calcium nitrate, calcium chloride, calcium hydroxide, calcium peroxide, calcium carbonate, potassium phosphate, dipotassium hydrogen phosphate, potassium dihydrogen phosphate, sodium phosphate, disodium hydrogen phosphate, sodium dihydrogen phosphate, or mixtures of these materials.

To further enhance the activity of this composition used in devices of the present invention, amorphous calcium phosphate or derivatives thereof, or compounds that are formed from a reaction of an amorphous calcium phosphate, can also be used in the composition. Examples include amorphous calcium phosphate fluoride (ACPF), amorphous calcium carbonate phosphate (ACCP), amorphous calcium carbonate phosphate fluoride (ACCPF), amorphous strontium phosphate (ASP), amorphous strontium calcium phosphate (ASCP), amorphous strontium calcium carbonate phosphate (ASCCP), and amorphous strontium calcium phosphate fluoride (ASCPF).

The amorphous calcium phosphate can also include a polypeptide, like a casein phosphopeptide, or a derivative of the casein phosphopeptide. In other embodiments, the amorphous calcium phosphate is stabilized with a synthetic oligomer or polymer, an anion, a cation, polyphosphate, a pyrophosphate, an amino acid, or compatible mixtures of any of these.

The amorphous calcium phosphate may be stabilized, such as a metal-or metalloid-stabilized calcium phosphate. Examples of metal-stabilized or metalloid-stabilized are those amorphous calcium phosphates that include the elements of, for example, zirconium, silicon, titanium, or mixtures of these. Specific examples of metal-stabilized or metalloid-stabilized calcium phosphates include zirconium-modified amorphous calcium phosphate (Zr-ACP), titanium-modified amorphous calcium phosphate (Ti-ACP), silicon-modified amorphous calcium phosphate (Si-ACP), amorphous calcium phosphate fluoride (ACPF) and amorphous calcium carbonate phosphate (ACCP). Magnesium and other divalent cations can also be used to enhance stability of ACP and its derivatives.

Crystalline, or semi-crystalline calcium phosphates may also be added to the compositions used in devices of the present invention. Specific examples include mono-, di-, tri-, alpha-tri-, beta-tri-, and tetra-calcium phosphate, hydroxyapatite (HAP), and mixtures thereof.

Embodiments of the present invention may have one or more of the following advantages. The compositions, devices and methods allow a user to carry out tooth mineralization simultaneously with tooth whitening. The devices reduce irritation and/or burning of gums and/or sensitive cheek tissues opposite tooth surfaces. The compositions allow for a high bioavailability of calcium and phosphate ions, leading to particularly efficient mineralization. The compositions can harden teeth and can reduce tooth sensitivity and/or reduce dental porosity and dental caries. Further, this composition may be efficient in stain prevention and extrinsic stain removal. Whitening can be rapid, occurring in less than 120 minutes, less than 90 minutes, less than 60 minutes, less than 45 minutes, less than 30 minutes, less than 15 minutes, or even less than 5 minutes.

Other aspects, features, and advantages will be apparent from the description and drawings, and from the claims.

The tooth-bleaching agent can be a peroxide, a metal chlorite, a perborate, a percarbonate, a peroxyacid, a persulfate, a carbamide, a hypochlorite, chlorine dioxide, a precursor to any of the just described whitening agents, or compatible mixtures of any of these materials. For example, the tooth-bleaching agent can include hydrogen peroxide, a complex thereof, such as a polymer adduct of hydrogen peroxide, or a source thereof.

The aqueous instability of bioactive glasses limits their use and effectiveness in oral care compositions. So, the compositions of the present invention are used in substantially anhydrous forms, such as dental strips, comprising less than about 5 percent by weight water, or less than about 2 percent by weight water, or even less than 1 percent by weight water.

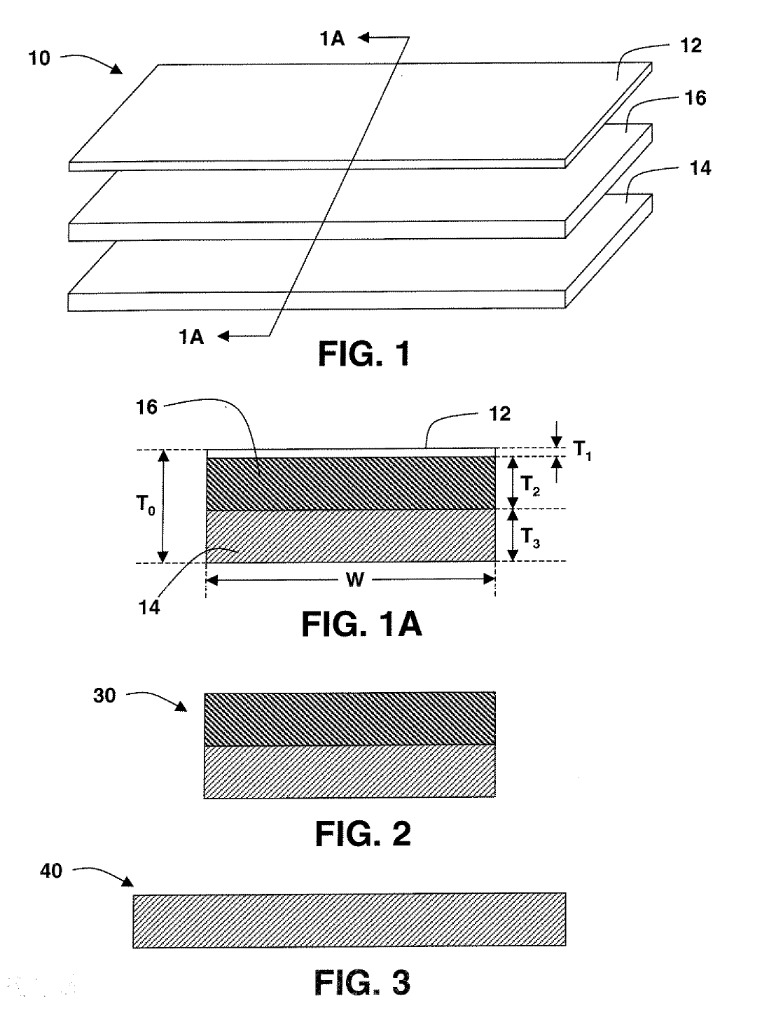

Referring to FIGS. 1 and 1A, a flexible dental strip 10 formed from a polymeric material includes a first layer 12, configured to contact one or more teeth, that includes a bioactive glass, a third layer 14, configured to contact sensitive cheek tissues opposite tooth surfaces, and a second layer 16 disposed between the first 12 and third 14 layers. The second layer includes a tooth-bleaching agent and a bioactive glass. In some embodiments, third layer 14 provides barrier properties for unidirectional flow of actives toward tooth surfaces, thus minimizing irritation to cheeks opposite tooth surfaces. In some embodiments, the tooth-bleaching agent and the bioactive glass may be maintained apart from one another, such as in different layers, prior to application of the device to the teeth. Once the device is contacted with the teeth, both the bioactive glass material and the tooth-bleaching agent subsequently contact the teeth. In certain embodiments, when making the strip, water is avoided, and the strip is maintained under anhydrous conditions until use to reduce reversion of the bioactive glass to an insoluble form and/or to maintain the efficacy of the tooth-bleaching agent.

In some embodiments, the flexible dental strip 10 is non-dissolvable and adherent to the teeth. For example, it is advantageous for the strip to adhere to the teeth for a time of between about 1 minute and 8 hours, or between 15 minutes and 4 hours, or between 30 minutes and 2 hours.

The amount of a bioactive glass in the strip generally ranges from about 0.5 percent by weight to about 40 percent by weight of the strip, or between about 10 percent by weight to about 35 percent by weight, or between about 10 percent by weight and 25 percent by weight. The amount of bleaching agent in the strip generally ranges from about 0.5 percent by weight to about 50 percent by weight, or between about 5 percent by weight to about 35 percent by weight, or even between about 5 percent by weight to about 20 percent by weight.

In embodiments when it is desired not to have a layer swell or dissolve in the oral cavity, non-swellable or non-dissolvable materials, such as polymers, are selected for the layer. Examples of such materials include hydroxypropylmethyl cellulose derivatives and copolymers, phthalates, succinates, acetates, and silicone waxes and polymers.

In some embodiments, it is advantageous that the strip be flexible so that it can conform to the many complicated and uneven surfaces of the teeth. This can be accomplished, for example by using a polymer that has a room temperature flexural modulus less than 100,000 psi, or less than 50,000 psi, or less than 25,000 psi, as measured using a dynamic mechanical analyzer. Flexibility can also be imparted by using a polymeric material having a Shore A hardness of less than 100 Shore A, or less than 75 Shore A, or less than 50 Shore A. Additional flexibility can be imparted by adding a plasticizer. The plasticizer can be incorporated into the strip at a concentration between about 0.1 percent and about 30 percent by weight of the strip. Specific examples of plasticizers include polyols, such as polyethylene glycol, triethyl citrate, propylene glycol, glycerin, sorbitol, glycerol acetate or mixtures of these plasticizers.

In some embodiments, dental strip 10 can include a flavoring agent, a fragrance and/or a sweetener. Flavoring agents include natural or synthetic essential oils, as well as various flavoring aldehydes, esters, alcohols, and other materials. Examples of essential oils include oils of spearmint, peppermint, wintergreen, sassafras, clove, sage, eucalyptus, marjoram, cinnamon, lemon, lime, grapefruit, and orange. The flavoring agent, fragrance and/or sweetener can be incorporated into the strip composition at a concentration of between about 0.1 percent and about 10 percent by weight, or between about 0.1 percent to about 2 percent by weight.

Coloring agents, such as pigments and dyes, can be included in the strips. For example, in some instances, it is desirable to have a colored strip that provides a visual cue to the user that it is time to remove the strip. The colored strip can be used in combination with a chemical or bio-sensor.

In some embodiments, the tooth whitening agent and the strip further includes a peroxide activator. Peroxide activators can increase the bleaching efficacy of peroxide components. Examples of peroxide activators include metals like iron, copper, or manganese, pH modifiers like hydroxide salts, zeolites, and photosensitizers such as titanium dioxide.

The strip can also include ingredients such as enzymes, malodor controlling agents, cleaning agents, such as phosphates, anti-staining agents, antibacterial agents, antigingivitis agents, anticaries agents, antiperiodontitis agents and tooth sensitivity agents. Examples of agents include cetylpyridinium chloride, chlorhexidine, zinc chloride and potassium nitrate.

In some embodiments, each layer 12, 14 and 16 of strip 10 is formed from a different polymeric material.

The strip can be formed by casting the polymeric layers. The strip can also be formed by extrusion or co-extrusion, or by molding or co-molding.

The rate at which the bleaching agent and/or the bioactive glass is solubilized (in the case of a solid bleaching agent), and subsequently released to a tooth surface can be controlled by properties such as, but not limited to, the film thickness, film texture, presence of film perforations, polymer properties, such as the structure, molecular weight, type, hardness and flexural modulus, properties of the whitening agent, properties of the bioactive glass, and the concentration of the bleaching agent and bioactive glass.

The thicknesses of the film layer may affect the residence time of any ingredients in the strip, including residence time of the bleaching agent and bioactive glass. The residence time can, for example, be determined by a combination of the composition of the polymer and the thickness of the film.

In some embodiments, it is advantageous to minimize the overall thickness of the strip since overall thickness can influence the mouth feel of the product, which can relate to user acceptance and compliance. Additionally, the thickness of the film can also affect its ability to conform and adhere to teeth in an efficient manner.

Referring particularly to FIG. 1A, an overall thickness To of the strip is from about 0.01 millimeters to about 2 millimeters, or from about 0.2 millimeters to about 1.5 millimeters; a thickness T₁ of first layer 12 from about 0.01 millimeters to about 0.5 millimeters, or from about 0.02 millimeters to about 0.3 millimeters; a thickness T₂ of second layer 16 from about 0.05 millimeters to about 1 millimeters, or from about 0.1 millimeters to about 0.8 millimeters; a thickness T₃ of third layer 14 from about 0.05 millimeters to about 1 millimeters, or from about 0. 1 millimeters to about 0.8 millimeters; and a width of the strip W from about 4 millimeters to about 40 millimeters, or from about 10 millimeters to about 35 millimeters, or from about 10 millimeters to about 25 millimeters.

In a specific embodiment, first and second layers 12 and 16 are cast from polyvinylpyrrolidone, and third layer 14 is formed from hydroxypropylmethylcellulose phthalate (HPMCP) having a molecular weight that is greater than 100,000, as measured relative to mono-disperse polyethylene glycol standards in tetrahydrofuran as solvent. This provides for a nearly impermeable third layer 14 and substantially prevents the whitening agent from reaching the sensitive cheek tissues that are opposite the tooth surfaces.

Strip 10 can be used, for example, to simultaneously remineralize and whiten teeth. In use, strip 10 is oriented such that layer 12 contacts the teeth of the user. Layer 14 then protects gums and sensitive cheek tissues from being exposed to the bleaching agent that is in layer 16. After contact is made, the bioactive glass diffuses from strip 10, as does the bleaching agent, toward the tooth surfaces. Contact with the teeth is maintained until the desired whitening result is obtained. It may be necessary to repeat the steps with a fresh strip 10 to obtain the desired whitening effect. In some embodiments, sufficient time is less than 30 minutes, or less than 15 minutes, 10 minutes or less than 5 minutes. Effective mineralization is also achieved using this composition/device containing bioactive glass and a hydrogen peroxide source. Without intending to bound by any particular theory, it is believed that the hydrogen peroxide molecules create a microenvironment that mimics the “natural” carbonic acid chemistry, and facilitates remineralization using soluble Ca²⁺ and PO₄³⁻ ions. It is believed that delivering Ca²⁺ and PO₄³⁻ ions in this manner can remineralize the enamel and whiten teeth.

The composition can be removed by rinsing or brushing. Alternatively, when the composition includes a water-soluble polymeric material, the composition, in time, will simply dissolve away.

As seen in FIG. 2, strip 30 includes barrier layer 34 and whitening layer 32. Whitening layer 32 includes both the whitening agent and the bioactive glass contained therein, for contacting teeth when the device is applied thereto. Barrier layer 32 may include a polymeric material for providing protection to the gum and cheek tissue, as well as flavoring agents and the like.

As seen in FIG. 3, a single layer strip 40 contains both the whitening agent and bioactive glass, as well as a polymer to maintain the strip in contact with the teeth under conditions effective to whiten and mineralize the teeth.

Generally, when making the compositions, water is avoided, and the compositions are maintained under anhydrous conditions until use to reduce reversion of the bioactive glass to an insoluble form and/or to maintain the potency of the whitening agent. In some embodiments, the initial total amount of water in composition is less than 1 percent by weight, less than 0.5 percent by weight, less than 0.25 percent by weight, less than 0.1 percent by weight, or less than 0.05 percent by weight.

In another embodiment, the flexible dental strip is a disintegrable film compositions comprising at least one water insoluble polymer, a disintegration facilitator selected from the group consisting of a plasticizer, a water insoluble particulate or mixtures thereof and, optionally, at least one topical or systemic active wherein the disintegrable film is partially, substantially or completely disintegrable in an aqueous environment. The disintegrable film composition of the present invention can be used as a single layer film or in conjunction with one or more additional film layers to form a bi- or multi-layered film product.

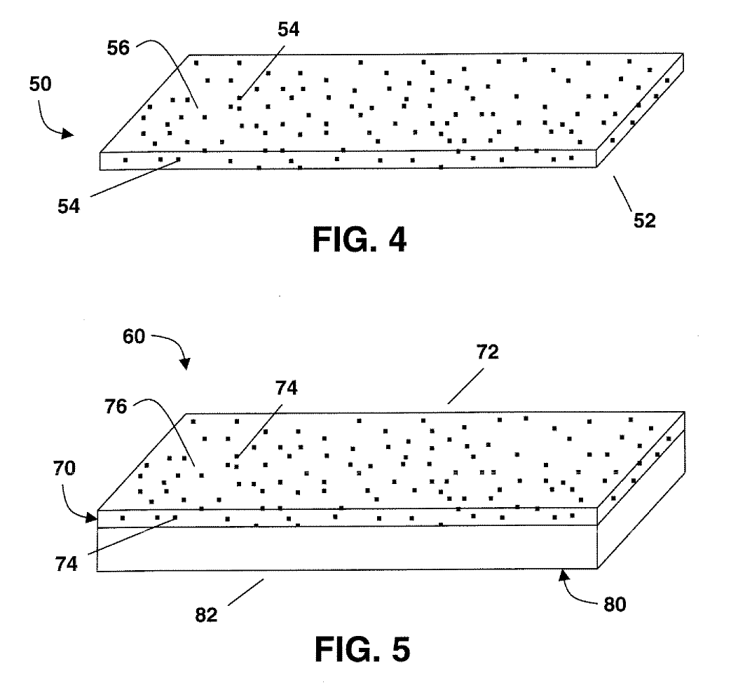

FIG. 4 shows a single layer disintegrable dental strip embodiment of the present invention. The figure shows strip 50 comprised of water insoluble polymer 52 and disintegration facilitator 54. Disintegration facilitator 54 may be fully incorporated into water insoluble polymer 52, may be only at the surface 56 of strip 50, or, as shown in the present embodiment, may be both fully incorporated into water insoluble polymer 52, as well as present at surface 56 of strip 50. FIG. 4 shows disintegration facilitator 54 uniformly distributed in water insoluble polymer 52. However, it is to be understood that the distribution of disintegration facilitator 54 in insoluble polymer 52 may be non-uniform. More or less disintegration facilitator 54 may be present at surface 56 of film 50 or in the bulk of insoluble polymer 52.

FIG. 5 shows film 60 comprised of first layer 70 and second layer 80. First layer 70 is comprised of water insoluble polymer 72 and disintegration facilitator 74. Disintegration facilitator 74 may be fully incorporated into water insoluble polymer 72, may be only at the surface of first layer 70, or, as shown in the present embodiment, may be both fully incorporated into water insoluble polymer 72, as well as present at surface 76 of first layer 70. Second layer 80 can be comprised of polymer. In one another embodiment, second layer 80 is an oral care active in a water soluble polymer 82 film layer such as that described in U.S. Pat. No. 6,596,298 to Leung et al. and U.S. Pat. No. 6,419,903 to Xu et al., both of which are herein incorporated by reference in their entirety. The bi-layer film is then applied to the teeth, oral mucosa or other affected area of the skin or mouth and allowed to disintegrate over time in the presence of saliva or other aqueous media and the mechanical action of the lips, tongue and other soft tissue.

The disintegrable film compositions of the present invention also comprise at least one disintegration facilitator selected from the group consisting of plasticizers or plasticizing agents, water insoluble particles or mixtures thereof.

Examples of suitable plasticizers include, but are not limited to, citric acid alkyl esters, glycerol esters such as glycerol monooleate and glycerol monostearate, phthalic acid alkyl esters, sebacic acid alkyl esters, sucrose esters, sorbitan esters, acetylated monoglycerides, glycerols, fatty acid esters, glycols, propylene glycol, and polyethylene glycols 200 to 12,000 and mixtures thereof. Specific plasticizers include, but are not limited to, lauric acid, triethyl citrate, acetyl triethyl citrate, triacetin (glyceryl triacetate), poloxamers, alkyl aryl phosphates, diethyl phthalate, tributyl citrate, dibutyl phthalate, dibutyl sebacate, polysorbate, as well as the series of polyethylene glycols sold under the tradename CARBOWAX (Union Carbide Corporation) and mixtures thereof.

In certain embodiments, the plasticizers can include mixtures of mono- and di-oleates supplied under the tradename ATMOS (Atmos 300) by American Ingredients, Kansas City, Mo.; triglycerides of caprylic/capric acids such as those sold by under the tradename NEOBEE (Neobee 1053) by Stepan, Chicago, Ill. and mono- and di-glycerides of edible fats or oils supplied by Lonza Inc., Fair Lawn, N.J. or Eastman Triacetin (food grade) supplied by Eastman Chemical Company, Kingsport, Tenn. The Atmos 300 and Neobee 1053 also function as releasing agents which aid in removal of the disintegrable layer from the casting sheet (e.g., polypropylene sheets) on which the layer is cast for drying.

When incorporated in the disintegrable film compositions of the present invention, the plasticizer is present at a concentration of from about 0.1 percent to about 10 percent, preferably from about 0.1 percent to about 5 percent, and most preferably from about 0.5 percent to about 1.5 percent by weight of the wet film composition.

The disintegration facilitator can also be a water insoluble particle. Various kinds of organic powders and inorganic powders can be used as the water-insoluble particles.

The inorganic powders which are useful herein include, but are not limited to, microfine particles or granules of alumina, talc, magnesium stearate, titanium dioxide, barium titanate, magnesium titanate, calcium titanate, strontium titanate, zinc oxide, silica sand, clay, mica, tabular spar, diatomaceous earth, various inorganic oxide pigments, chromium oxide, cerium oxide, antimony trioxide, magnesium oxide, zirconium oxide, barium sulfate, barium carbonate, calcium carbonate, silica (colloidal or fumed), silicon carbide, silicon nitride, boron carbide, titanium carbide, and mixtures thereof.

The organic powders that are useful herein include cross-linked and non-cross-linked polymer powders, organic pigments, charge controlling agents, and waxes, for example. The cross-linked and non-cross-linked resin powders include, but are not limited to, resin powders of the styrene type, acrylic type, methacrylic type, polyethylene type, polypropylene type, silicone type, polyester type, polyurethane type, polyamide type, epoxy type, polyvinyl butyral type, for example. Mixtures of any of the above organic or inorganic powders can also be used. Additional particles useful in the present invention can be found in U.S. Pat. No. 6,475,500; U.S. Pat. No. 5,611,885; and U.S. Pat. No. 4,847,199 the contents of each which are herein incorporated by reference in its entirety.

The water insoluble particles of the present invention generally have a particle size or aggregate particle size of less than 10 microns, optionally, from about 0.01 microns to about 5 microns, optionally, from about 0.1 microns to about 1 micron, and, optionally, from about 0. 1 to about 0.5 microns.

In certain embodiments, the insoluble particles can include Cabosil M-5 (fumed untreated silica) supplied by Cabot, Tuscola, Ill.

When incorporated in the disintegrable film compositions of the present invention, the water insoluble particle is present at a concentration of from about 0. 1 percent to about 20 percent, optionally, from about 0.5 percent to about 10 percent, and, optionally, from about 1 percent to about 7 percent by weight of the film composition.

Where the disintegrable film of the present invention forms the first or backing layer of a multi-layer or bi-layer film, the thickness of the disintegrable film layer can optionally range from about 1 micron to about 20 microns, optionally from about 3 microns to about 15 microns, optionally from about 5 microns to about 12 microns. The thicknesses of any additional layers can equal the range of thickness of the first or backing layer or range from about 30 microns to about 150 microns, optionally from about 45 microns to about 130 microns, optionally from about 70 microns to about 120 microns.

### EXAMPLES

Polyvinylpyrrolidone (PVP K90) having a molecular weight of about 90,000 and polyvinylpyrrolidone (PVP)-hydrogen peroxide complex (Peroxydone K 90) were both obtained from ISP (Wayne, N.J.); Bioactive glass (45S5) was obtained from MO-SCI Health Care L.L.C. (Rolla, Mo.), Three grades of hydroxypropylmethylcellulose phthalate (HPMCP) (HP-50, -55, and -55S) were from ShinEtsu (Tokyo, Japan); and Carbopol (974, 980, 956) were obtained from Lubrizol Advanced Materials, Inc., (Cleveland, Ohio). Each was used as received, without additional purification.

### Example 1

### Two Layer Oral Care Strip with Bioactive Glass and Whitening Agent in a Single Layer

The first layer (active layer) was cast from the following formulation:

| Ingredient | w/w % |
|---|---|
| Ethanol (200 proof) | 61.20 |
| Peroxydone K-90 | 35.00 |
| PEG-400 | 0.50 |
| Menthol | 0.15 |
| Sodium Saccharin | 0.15 |
| Bioactive glass 45S5 | 3.00 |
| Total | 100.00 |

The second layer (barrier layer) was cast from the following formulation:

| Ingredient | w/w % |
|---|---|
| Ethanol (190 proof) | 69.80 |
| HPMCP | 18.00 |
| DI Water | 10.00 |
| PEG-1000 | 1.20 |
| Double Mint Oil base | 1.00 |
| Total | 100.00 |

To make the dental strip, the barrier layer was first cast onto a Teflon substrate. After drying and loss of volatiles, the active layer was then cast on top of the barrier layer and allowed to dry. If needed, another layer can be cast on top these two layers. During the formation of the strip, water is generally avoided to prevent loss of activity of bioactive glass.

The two-layer strip was tested using X-ray diffraction (XRD), FTIR, and ICP. The results show typical signatures of bioactive glass, which on hydration exhibited formation of hydroxyapatite type material. Testing also indicated that the strip released calcium ions in-vitro. Furthermore, the strip bleached tooth specimens, increased the hardness of tooth specimens, and increased mineral volume fraction of tooth specimens. In this composition the stabilities of both the bleaching active and the bioactive glass were acceptable. Further, the in-vitro bleaching data suggest enhanced efficacy of this combination in reducing the intensity of surface stains. It is hypothesized that the relative increase in pH caused by hydration of bioactive glass enhances peroxide activation, which in turn may result in improved efficiency of products containing this combination.

### Example 2

### Three Layer Oral Care Strip with Bioactive Glass and Whitening Agent in Separate Layers

The first layer (active layer) was cast from the following formulation:

| Ingredient | w/w % |
|---|---|
| Ethanol (200 proof) | 93.20 |
| PVP K-90 | 5.00 |
| PEG-400 | 0.50 |
| Menthol | 0.15 |
| Sodium Saccharin | 0.15 |
| Bioactive glass 45S5 | 2.00 |
| Total | 100.00 |

The second layer (Bioactive glass layer) was cast from the following formulation:

| Ingredient | w/w % |
|---|---|
| Ethanol (200 proof) | 61.87 |
| Peroxydone K-90 | 31.43 |
| PEG-400 | 3.00 |
| PEG-4600 | 0.50 |
| Menthol | 0.15 |
| Sodium Saccharin | 0.15 |
| Bioactive glass 45S5 | 3.00 |
| Total | 100.00 |

The third layer (barrier layer) was cast from the following formulation:

| Ingredient | w/w % |
|---|---|
| Ethanol (190 proof) | 69.80 |
| HP-55S | 18.00 |
| DI Water | 10.00 |
| PEG-1000 | 1.20 |
| Flavor Oil | 1.00 |
| Total | 100.00 |

The strips were made by casting according to the procedure described in Example 1.

### Example 3

### A Single Layer Oral Care Strip

The strip was cast from the following formulation:

| Ingredient | w/w % |
|---|---|
| Ethanol (200 proof) | 49.50 |
| Peroxydone K-90 | 30.43 |
| HP55S | 12.57 |
| PEG-400 | 2.00 |
| Menthol | 0.50 |
| Sodium Saccharin | 0.50 |
| Bioactive glass 45S5 | 4.50 |
| Total | 100.00 |

The strip was made by casting according to the procedure described in Example 1.

### Example 4

### Two Layer Oral Care Strip with Bioactive Glass and Whitening Agent in Different Layers

The first layer was cast from the following formulation:

| Ingredient | w/w % |
|---|---|
| Gums | 0.3 |
| Pullulan | 3.00 |
| Plasdone, K-90 (USP) | 14.00 |
| Sodium Saccharin (USP) | 0.50 |
| PEG 3350 NF | 0.50 |
| Potassium monobasic | 0.26 |
| Potassium dibasic | 0.09 |
| H2O | 64.97 |
| Peroxide | 12.72 |
| Mint Flavor | 2.50 |
| Polysorbate 20 NF | 0.35 |
| ATMOS 300 | 0.35 |
| Glycerine | 0.50 |
| Total | 100.00 |

The second layer was cast from the following formulation:

| Ingredient | w/w % |
|---|---|
| Pharmaceutical Glaze | 54.83 |
| Bioactive glass 45S5 | 4.01 |
| Ethanol USP, 200 Proof | 35.67 |
| Eastman Triacetin, Kosher | 0.25 |
| Caprylic Capric | 0.50 |
| Triglyceride | |
| ATMOS 300 | 4.75 |
| Total | 100.00 |

The strips were made by casting according to the procedure described in Example 1.

### Peroxide Stability:

Whitening strips as prepared in Example 1 were evaluated for peroxide stability versus a comparable whitening strip containing H₂O₂, but with no bioactive glass, by placing them in a chamber at 40° C./75% RH, for thirteen weeks. Peroxide content was measured on a weekly basis using standard ceric sulfate protocol and reported as total peroxide (mg). Results are shown in Table 1.

| Time | | |
|---|---|---|
| (weeks) | Example 1 | Comparative |
| 0 | 21.94 | 21.89 |
| 4 | 15.71 | 16.74 |
| 8 | 13.83 | 14.84 |
| 10 | 12.68 | 15.762 |
| 13 | 11.92 | 13.67 |

As shown, strips according to the present invention maintained comparable stability when compared to strips containing no bioactive glass.

### Peroxide Release:

Effect of bioactive glass on peroxide release from the strips as a function of time was studied by placing a whitening strip onto the surface of a petridish with the peroxide layer of the strip facing upwards. Three milliliters of synthetic saliva was added to each petridish and the petridish was quickly placed on top of an orbital shaker. Samples were collected at regular intervals and analyzed for percent peroxide released at the noted intervals.

| | H2O2 + 2.8% | H2O2 + 7.5% | H2O2 |
|---|---|---|---|
| Time | bioactive glass | bioactive glass | 0% bioactive glass |
| (min) | % Released | % Released | % Released |
| 0 | 0.000 | 0.000 | 0.000 |
| 3 | 34.730 | 29.232 | 20.547 |
| 6 | 44.480 | 42.420 | 27.008 |
| 9 | 41.579 | 51.054 | 22.002 |
| 12 | 47.381 | 43.780 | 35.856 |
| 15 | 66.640 | 50.714 | 38.242 |
| 20 | 56.890 | 53.637 | 44.587 |
| 25 | 56.970 | 59.143 | 49.418 |
| 30 | 71.313 | 69.409 | 56.345 |
| 45 | 68.896 | 67.165 | 61.059 |

As shown, percent peroxide released from the strips including both H₂O₂ and bioactive glass increased compared to comparable strips containing no bioactive glass. Of particular note, the rate of peroxide release for the strips of the invention was greater than that of the comparative example over the first 15 minutes, indicating a greater release of peroxide radicals. Thus, more peroxide radicals delivered from the inventive strips are available for bleaching shortly after application to the teeth than are for the comparable example.

### In Vitro Bleaching of Human Teeth

Whitening efficacy of strips was measured using a tooth whitening efficacy model utilizing human teeth to measure the whitening effect of devices of the invention compared to conventional devices. The shade, i.e. the color, of the teeth was measured before and after a treatment using a calorimeter, such as the MHT SpectroShade System.

The difference in tooth shade before and after treatment is a measure of the efficacy of the device.

| Sample | Delta L | Delta a | Delta b |
|---|---|---|---|
| Comparative | 0.877 | −1.491 | −3.233 |
| Example 1 | 2.13 | 0.035 | −3.125 |

In-Vitro Bleaching Study (Human Teeth) Rate of Whitening with and without Peroxide:

Delta L Value

| | | H₂O₂ + | H₂O₂ + | | PVP + |
|---|---|---|---|---|---|
| | | 2.8% | 7.5% | | 7.5% |
| | H2O2 0% | bioactive | bioactive | | bioactive |
| Treatment | bioactive glass | glass | glass | PVP | glass |
| 0 | 0 | 0 | 0 | 0 | 0 |
| 3 | 1.15 | 1.46 | 1.3 | 0.59 | 0.01 |
| 7 | 1.96 | 2.15 | 1.83 | 0.44 | 0.4 |
| 10 | 3.29 | 3.39 | 3.25 | 0.74 | 0.92 |
| 14 | 3.13 | 3.61 | 3.41 | 1.11 | 0.74 |

Delta b Value

| | | H₂O₂ + | | | PVP + |
|---|---|---|---|---|---|
| | | 2.8% | | | 7.5% |
| | | bioactive | | | bioactive |
| Treatment | Comparative | glass | Example 1 | PVP | glass |
| 0 | 0 | 0 | 0 | 0 | 0 |
| 3 | −2.12 | −1.75 | −2.23 | −0.28 | −1.1 |
| 7 | −4.78 | −3.44 | −3.86 | −0.44 | −2.38 |
| 10 | −4.6 | −4.54 | −4.87 | −0.66 | −2.66 |
| 14 | −5.4 | −5.07 | −5.83 | −0.48 | −2.85 |

### Increase in Enamel Micro Hardness:

The purpose of this test was to evaluate in-vitro efficacy of soluble calcium and phosphate from bioactive glass to enhance hardness of enamel. Enamel specimens (3×3×3 mm diameter window) were removed from extracted human teeth and mounted on acrylic rods. The enamel surface was ground and polished using alumina slurry, rinsed with deionized water and air-dried. Artificial lesions were then formed in the specimens by a 96-hour immersion in a partially saturated HAP acid buffer consisting of 0.1 mM lactic acid and 0.2 wt % Carbopol C907 adjusted to a pH of 5. Four baseline indentations per specimen were placed with a Vickers diamond at a 200-gram load for 15 seconds and the indentation lengths were within a range from 25-45 microns. Enamel specimens (N=15 per treatment) were then cycled daily through a series of treatments with strip prototypes repeated over a 7 day time period. Artificial saliva was prepared fresh daily. After treatment, the enamel microhardness (reported as Knoops Hardness—HK) was assessed and the difference between initial and final microhardness was calculated as a measure of remineralization efficacy.

| Treatment | Av. Initial HK | Av. Final HK | % Change in HK |
|---|---|---|---|
| Comparative | 41.23 | 42.33 | 2.77 |
| Example 1 | 44.33 | 93.40 | 112.12 |
| H₂O₂ + 2.8% | 41.73 | 64.40 | 54.80 |
| bioactive glass | | | |

While embodiments of devices that have been described are in strip-form, embodiments are possible in which the oral care device is in other forms, e.g. in the form of a mouthpiece, e.g. a heat moldable mouthpiece that is configured to be worn by a user wherein an anhydrous coating or other bleaching material is placed into the mouthpiece.

What is claimed is:

1. An oral care device configured to contact teeth, the device comprising disposed therein:
a bioactive glass in an amount effective to mineralize teeth when contacted with said teeth under conditions effective to mineralize said teeth; and
a tooth-bleaching agent in an amount effective to whiten teeth when contacted with said teeth under conditions effective to whiten said teeth.

2. The oral care device of claim 1, wherein said bioactive glass comprises about 45 percent SiO₂, about 24.5 percent CaO, about 24.5 percent Na₂O, and about 6 percent P₂O₅.

3. The oral care device of claim 1, wherein said tooth-bleaching agent is selected from the group consisting of peroxides, metal chlorites, perborates, percarbonates, peroxyacids, persulfates, carbamides, hypochlorites and chlorine dioxide.

4. The oral care device of claim 2, wherein said tooth-bleaching agent comprises hydrogen peroxide, a complex thereof, or a source thereof.

5. The oral care device of claim 3 comprising from about 0.5 to about 40 percent by weight of said bioactive glass and from about 0.5 to about 50 percent by weight of said tooth-bleaching agent.

6. The oral care device of claim 5, wherein said device is in the form of a strip.

7. The oral care device of claim 6, wherein said strip comprises a polymeric material.

8. The oral care device of claim 7, wherein said polymeric material is selected from the group consisting polyvinylpyrrolidone, a cellulosic polymer, polyvinyl alcohol, hydroxyethyl cellulose, hydroxypropyl cellulose, hydroxypropylmethyl cellulose, sodium carboxymethyl cellulose, polyethylene oxide, polyethylene glycol, polyacrylic acid, silicone, hydroxypropylmethylcellulose phthalate, polyvinyl acetate, cellulose acetate phthalate, gelatin sodium alginate, carbomer, polyvinyl pyrrolidone-vinyl acetate copolymer, polyalkylvinylether-maleic acid copolymer, polypropylene glycol, polyethylene glycol-polypropylene glycol copolymers, quatemized copolymers, quaternized copolymer of vinylpyrrolidone and dimethylaminoethyl methacrylate.

9. The oral care device of claim 7, wherein said strip comprises a plurality of layers.

10. The oral care device of claim 9, wherein said strip comprises a first layer configured to contact said teeth, said first layer comprising said bioactive glass; a third layer; and a second layer disposed between said first and said third layers, said second layer comprising said tooth-bleaching agent.

11. The oral care device of claim 1 wherein said bio active glass and said tooth-bleaching agent are maintained apart from one another in said device prior to application of the device to said teeth.

12. The oral care device of claim 1 further comprising a stabilized amorphous calcium phosphate selected from the group consisting of zirconium-modified amorphous calcium phosphate, titanium-modified amorphous calcium phosphate, silicon-modified amorphous calcium phosphate, amorphous calcium phosphate fluoride and amorphous calcium carbonate phosphate.

13. The oral care device of claim 1 further comprising a crystalline or semi-crystalline calcium phosphate selected from the group consisting of mono-calcium phosphate, di-calcium phosphate, tri-calcium phosphate, alpha-tri-calcium phosphate, beta-tri-calcium phosphate, tetra-calcium phosphate and hydroxyapatite.

14. The oral care device of claim 1 wherein said bioactive glass comprises a biologically-active molecule bound thereto.

15. A method of simultaneously mineralizing and whitening teeth, the method comprising:
contacting said teeth with an oral care device comprising a bioactive glass in an amount effective to mineralize teeth when contacted with said teeth under conditions effective to mineralize said teeth, and a tooth-bleaching agent in an amount effective to whiten teeth when contacted with said teeth under conditions effective to whiten said teeth; and
maintaining contact of said oral care device with said teeth under said conditions effective to mineralize and whiten said teeth.

16. The method of claim 15 wherein said tooth-bleaching agent is selected from the group consisting of peroxides, metal chlorites, perborates, percarbonates, peroxyacids, persulfates, carbamides, hypochlorites and chlorine dioxide.

17. The method of claim 16 wherein said oral care device comprises from about 0.5 to about 40 percent by weight of said bioactive glass and from about 0.5 to about 50 percent by weight of said tooth-bleaching agent.

18. The method of claim 16 wherein said oral care device is maintained in contact with said teeth for at least about 5 minutes.

19. The method of claim 18 wherein said method is repeated from 2 to about 7 times per day.

20. The method of claim 17 wherein said oral care device is maintained in contact with said teeth for at least about 5 minutes.

21. The method of claim 20 wherein said method is repeated from 2 to about 7 times per day.

22. The method of claim 18 wherein said method is repeated over a time period of from 1 to about 28 days.

23. The method of claim 20 wherein said method is repeated over a time period of from 1 to about 28 days.
